(19) United States
(12) Patent Application Publication
(10) Pub. No.: US 2008/0171886 A1
(43) Pub. Date: Jul. 17, 2008

(54) Preparation Of An Atorvastatin Intermediate Using A Paal-Knorr Condensation

(75) Inventors: Benjamin Simon Davies, Dublin (IE); Alastair Alexander Denholm, Dublin (IE); Patrick Gerard Thomas Kelleher, Dublin (IE); William Eugene Tully, Dublin (IE)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 11/908,724

(22) PCT Filed: Jan. 3, 2006
(86) PCT No.: PCT/US06/00014

§ 371 Date: Sep. 14, 2007

(30) Foreign Application Priority Data
Jan. 6, 2005 (US) 11031307

(51) Int. Cl. C07D 207/30

(52) U.S. Cl. 548/561

(57) ABSTRACT

A plug-in Wi-Fi access point device and system is provided that is configured for Wi-Fi or other wireless network and to be directly plugged into a face plate/workstation. The plug-in Wi-Fi access point device and system advantageously provides ease of integration into an existing wired network without the use of a patch cord, security from removal by unauthorized personnel, non obtrusiveness in relation to other face plate/workstation jacks, and the ability to be powered through an Ethernet connection to avoid the need for a separate power source. Furthermore, the plug-in Wi-Fi access point device and system may beneficially permit the integration of complementary connections within the access point including, for example, data, voice, and video connection types. The plug-in Wi-Fi access point device includes a housing for accommodating Wi-Fi access point circuitry, Wi-Fi access point circuitry mounted within the housing, and a connector mounted on side of the housing for direct plug-in into an Ethernet jack of a face plate/workstation. The plug-in Wi-Fi access point device may further include a locking or self-locking mechanism, an integrated hub/switch/router, and at least one integrated voice, video and/or data jack for voice, video or data communication.

### REFERENCE TO RELATED APPLICATIONS

This patent application is a Continuation-in-Part of U.S. patent application Ser. No. 10/969,472, filed on Oct. 20, 2004, and claims priority to U.S. Provisional Patent Application No. 60/535,774, filed Jan. 12, 2004, both of which disclosures are hereby incorporated by reference as part of the present disclosure.

### FIELD OF THE INVENTION

The present invention relates to Wi-Fi access points. More particularly, the present invention relates to an advantageous device and system for providing Wi-Fi access. Still more particularly, the present disclosure relates to a plug-in Wi-Fi access point device and system that can be directly plugged into and integrated into an existing face plate or workstation.

### BACKGROUND OF THE INVENTION

As used herein, the term “Wi-Fi” is short for wireless fidelity and is meant to encompass any type of 802.11 network or other wireless networks, whether 802.11b, 802.11a, 802.11g, 802.16, dual-band, etc. The term “Wi-Fi” is currently promulgated by the Wi-Fi Alliance. Any products tested and approved as “Wi-Fi Certified” (a registered trademark) by the Wi-Fi Alliance are certified as interoperable with each other, even if they are from different manufacturers. A user with a “Wi-Fi Certified” product can use any brand of access point with any other brand of client hardware that also is certified. Typically, however, any Wi-Fi product using the same radio frequency (e.g., 2.4 GHz for 802.11b or 802.11g, and 5 GHz for 802.11a) will work with any other, even if such products are not “Wi-Fi Certified.” The term “Wi-Fi” is further intended to encompass future versions and/or variations on the foregoing communication standards. Each of the foregoing standards is hereby incorporated by reference.

Thus, a wireless access point is a bridge between a wired and a wireless network. Wireless access points function like a wireless hub connecting all the wireless devices together and then connecting them to a wired network. A wireless network access point is an essential part of a wireless network in that the access point facilitates connection to the Internet and/or another network, e.g., an intranet, extranet, WAN, LAN or the like. Many wireless access points are now built into wireless routers so that the features of a broadband router and a wireless access point are provided in one unit. Wireless access points generally have differing levels of performance, e.g., different wireless access points perform at varying data transmission speeds.

Many manufacturers are producing units that offer wireless access functionality. Thus, for example, the NETGEAR (Santa Clara, Calif.) wireless access points have been being built into broadband routers. LINKSYS (Irvine, Calif.), D-LINK (Fountain Valley, Calif.) and BELKIN (Compton, Calif.) also manufacture wireless broadband routers that include a built-in wireless access point.

Wireless access points are now appearing in what may be termed “hot spots” in hotels, train stations and airports. These access points are making wireless Internet connectivity available to travelers/individuals who can connect to the Internet or a desired network, e.g., a corporate network via a virtual private network (VPN), through wireless communication technology.

As used herein, the term “access point” encompasses a hardware device and/or associated software that acts as a communication hub for users of a wireless device to connect to a wired network, e.g., a local area network (LAN). Conventional access points generally provide a predetermined level of security for wireless communications that pass through the access point, and extend the physical range of service to which a wireless user has access.

Existing 802.11 access points suffer from various limitations and/or drawbacks. For example, current Wi-Fi access points are generally bulky and obtrusive, need to be connected via a patch cord, and often require an external power cord. Moreover, conventional Wi-Fi access ports are difficult to integrate into a desired environment because they require installation brackets, and boxes, which frequently results in a non-desirable and/or unacceptable physical presence in the environment. In addition, conventional Wi-Fi access points may be susceptible to tampering including being easily removable by unauthorized personnel.

U.S. patent application Ser. No. 10/969,472 filed on Oct. 20, 2004, discloses a Wi-Fi access point device and system for integrating operative aspects of a Wi-Fi access point into a wired wall network using a patch cord. The integration of the Wi-Fi access point into a standard switch and outlet boxes and/or standard wall plates requires the removal of a face plate and the associated time to integrate the Wi-Fi access point into the outlet box and/or wall plate.

A need exists for improved devices that provide Wi-Fi access point device and system and that overcome the foregoing limitations and/or drawbacks.

### SUMMARY OF THE DISCLOSURE

The present disclosure is distinguishable over the prior art in that it provides for a streamlined, easy to install, non-obtrusive, tamper resistant, direct plug-in WiFi access point device and system. The plug-in WiFi access point device and system does not require a patch cord to connect the access point to the wired network and may be powered through the Ethernet, such as to avoid the need for separate power.

One aspect of the present disclosure is directed to a Wi-Fi access point device and system that offers unobtrusive plug-in connectivity to a wall jack wired for network connectivity, is quickly and easily integrated into a desired environment, and is powered through the Ethernet. The Wi-Fi access point device and system includes a housing means for accommodating Wi-Fi access point circuitry; a Wi-Fi access point circuitry mounted with respect to said housing means; and a connector means mounted with respect to said housing means, wherein said connector means is dimensioned for direct plug-in into an Ethernet jack of a face plate/workstation for establishing a network communication.

A further aspect of the present disclosure is directed to a Wi-Fi access point device and system that offers unobtrusive plug-in connectivity to a wall jack wired for network connectivity, is quickly and easily integrated into a desired environment, is powered through the Ethernet, and does not decrease the number of jacks available at the face plate or workstation. The Wi-Fi access point device and system includes a housing means for accommodating Wi-Fi access point circuitry; a Wi-Fi access point circuitry mounted with respect to said housing means; a connector means mounted with respect to said housing means, wherein said connector means is dimensioned for direct plug-in into an Ethernet jack of a face plate/workstation for establishing a network communication; and at least one communication element mounted with respect to said housing means, wherein said at least one communication element is selected from the group consisting of: (i) one or more voice jacks, (ii) one or more video jacks, (iii) one or more data jacks, and (iv) a combination thereof.

Another aspect of the present disclosure is directed to a Wi-Fi access point and system that offers unobtrusive plug-in connectivity to a wall jack wired for network connectivity, is quickly and easily integrated into a desired environment, is powered through the Ethernet, and. is tamper resistant. The Wi-Fi access point device and system includes a housing means for accommodating Wi-Fi access point circuitry; a Wi-Fi access point circuitry mounted with respect to said housing means; a connector means mounted with respect to said housing means, wherein said connector means is dimensioned for direct plug-in into an Ethernet jack of a face plate/workstation for establishing a network communication; and a locking or self-locking means for securing said device within said face plate/workstation.

Another aspect of the present disclosure is directed to a Wi-Fi access point and system that offers unobtrusive plug-in connectivity to a wall jack wired for network connectivity, is quickly and easily integrated into a desired environment, is powered through the Ethernet, is tamper resistant, and does not decrease the number of jacks available at the face plate or workstation. The Wi-Fi access point device and system includes a housing means for accommodating Wi-Fi access point circuitry; a Wi-Fi access point circuitry mounted with respect to said housing means; a connector means mounted with respect to said housing means, wherein said connector means is dimensioned for direct plug-in into an Ethernet jack of a face plate/workstation for establishing a network communication; at least one communication element mounted with respect to said housing means, wherein said at least one communication element is selected from the group consisting of: (i) one or more voice jacks, (ii) one or more video jacks, (iii) one or more data jacks, and (iv) a combination thereof; and a locking or self-locking means for securing said device within said face plate/workstation.

Another aspect of the present disclosure relates to a method of providing a Wi-Fi access point system that offers unobtrusive plug-in connectivity to a wall jack wired for network connectivity, is quickly and easily integrated into a desired environment, and is powered through the Ethernet. The method of providing a Wi-Fi access point device and system includes the steps of: (a) providing a plug-in Wi-Fi access point device including a housing, Wi-Fi access point circuitry, and one or more antenna in electronic communication with the Wi-Fi access point circuitry and a RJ45 plug mounted on said housing; (b) providing a face plate/workstation including at least one Ethernet jack for network connectivity, wherein said at least one RJ45 jack; (c ) connecting said RJ45 plug mounted on said housing to said at least one RJ45 jack of said face plate/workstation; (d) initializing said plug-in Wi-Fi access point device with parameters for connecting a wireless device to said Wi-Fi access point system.

Numerous advantages result from the plug-in Wi-Fi access point device and system disclosed herein and the uses/applications therefore.

For example, in exemplary embodiments of the present disclosure, the plug-in Wi-Fi access point device and system provides for the removal of the patch cord that is typically required to connect the access point to the wired network.

In a further exemplary embodiment of the present disclosure, the plug-in Wi-Fi access point device and system disclosed provides a discrete, visibly appealing access point with respect to the front of the wall or the front of existing face plate/workstation for plug-in.

In an alternative embodiment of the present disclosure, the plug-in Wi-Fi access point device and system disclosed is free from any exposed wired network ports or jacks so as to facilitate more efficient dimensioning and compact sizing of the overall assembly.

A further advantage associated with the exemplary embodiments of the disclosed plug-in Wi-Fi access point device and system is that it provides for direct connection of the access point to the wired network via a connector integrated within the access point.

A further advantage associated with the exemplary embodiments of the disclosed plug-in Wi-Fi access point device and system is that is provides for improved ease of integration into an existing wired network via one of more RJ45 jacks of an existing face plate or workstation.

A further advantage associated with the exemplary embodiments of the disclosed plug-in Wi-Fi access point device and system is that is does not disturb adjacent ports of the existing face plate/workstation that it plugs into. The size/dimension of the disclosed plug-in Wi-Fi access point device and system is readily selected so as to be unobtrusive to other jacks mounted into conventional commercial/residential face plates (e.g., decorator face plates and the like).

A further advantage associated with the exemplary embodiments of the disclosed plug-in Wi-Fi access point device and system is that it provides enhanced security and tamper resistance by being removable only by authorized personnel via the use of a specific tool or mechanism to unplug it from existing face plate/workstation.

A further advantage associated with the exemplary embodiments of the disclosed plug-in Wi-Fi access point device and system is that it may provide one or more integrated voice, video and/or data communication jacks, and hence after plug-in into the jack of the faceplate/workstation does not decrease the number of jacks available.

Still a further advantage associated with the exemplary embodiments of the disclosed plug-in Wi-Fi access point device and system is that it is powered through the Ethernet connection so as to thereby avoid the need for a separate power source/supply.

Still a further advantage associated with the exemplary embodiments of the disclosed plug-in Wi-Fi access point device and system is that it has applicability for installations regardless of, and completely independent of, any applicable standards, whether now existing or developed in the future. Thus, the disclosed Wi-Fi access point devices and systems have applicability for any 802.11 network or other wireless networks, whether 802.11b, 802.11a, 802.11g, 802.16, dual-band, etc., but the disclosed devices/systems are expressly not limited to applications that conform to or satisfy such standards.

Additional advantageous features and functions associated with the disclosed plug-in Wi-Fi access point device and system will be readily apparent from the detailed description which follows, particularly when reviewed together with the drawings appended hereto.

### BRIEF DESCRIPTION OF THE FIGURES

To assist those of ordinary skill in the relevant art in making and using the subject matter hereof, reference is made to the appended drawings, wherein:

FIG. 1 is a perspective view of an illustrative embodiment of a plug-in Wi-Fi access point device according to the present disclosure.

FIG. 2 is an alternative perspective view of the illustrative embodiment of FIG. 1.

FIG. 3 is a perspective view of an alternative embodiment of a plug-in Wi-Fi access point device according to the present disclosure.

FIG. 4 is a perspective view of an illustrative embodiment of a plug-in Wi-Fi access point system according to the present disclosure.

### DETAILED DESCRIPTION OF EXEMPLARY EMBODIMENT(S)

According to the present disclosure, Wi-Fi access points devices/systems suitable for providing, inter alia, direct plug-in into a face plate/workstation are provided. The Wi-Fi access points, as disclosed herein, are well suited for integrating operative aspects of a Wi-Fi access point into the face plate of a wall or workstation having one or more RJ45 jacks. The disclosed plug-in Wi-Fi access point devices/systems have applicability for installations regardless of, and independent of, any applicable standards, whether now existing or developed in the future. Thus, the disclosed Wi-Fi access point devices/systems have applicability for any 802.11 network or other wireless networks, whether 802.11b, 802.11a, 802.11g, 802.16, dual-band, etc., but the disclosed devices/systems are expressly not limited to applications that conform to or satisfy such standards.

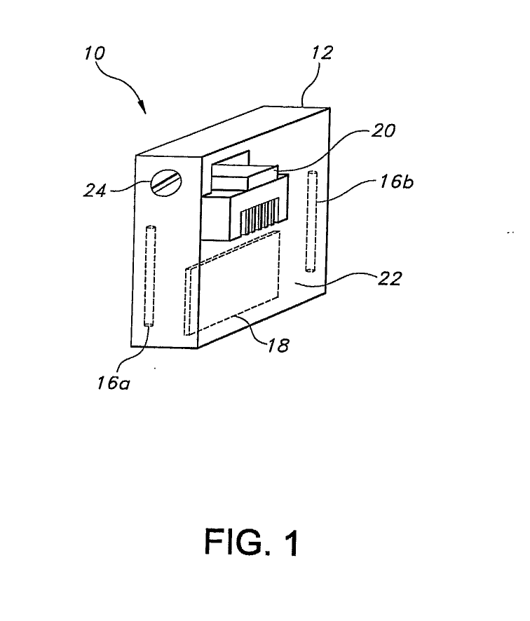

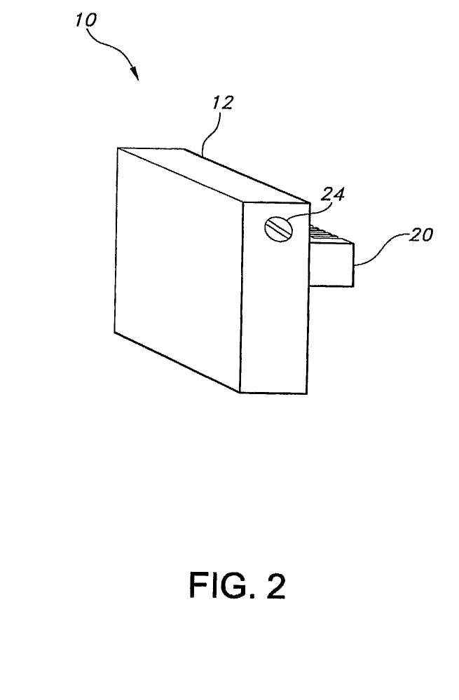

With reference now to the drawings and in particular to FIG. 1 and FIG. 2, there is shown by way of illustrative example a plug-in Wi-Fi access point device in accordance with the present disclosure generally represented by reference numeral 10. The plug-in Wi-Fi access point device 10, as shown, has a housing 12 suitable for accommodating Wi-Fi access point circuitry. The housing 12 means is advantageously configured and dimensioned for installation with respect to a face plate or workstation. In an exemplary embodiment, the housing 12 is rectangular in shape and sized so as to be unobtrusive to other jacks of a face plate/workstation where it is plugged into. The housing 12 may also optionally have vents (not shown) for dissipating heating generated within the housing 12.

The Wi-Fi access point circuitry, as shown in FIG. 1 in phantom, includes one or more antenna 16a, 16b, one or more substrates 18 (e.g., a predefined printed circuit board (PCB)), and an optional integrated hub/switch/router (not shown). Each antenna 16a, 16b, can be of conventional design and may be combined and/or supplemented to provide lesser/greater numbers of antenna without departing from the spirit or scope of the present disclosure. Each antenna 16a, 16b is mounted with respect to one or more PCB 18 that may be sized and shaped to be effectively accommodated by the housing 12 as well as to cooperate with a conventional face plate or workstation. For example, a diversity antenna may be used for the one or more antenna 16a, 16b mounted with respect to the one or more PCB 18. A plurality of electronic components may also be mounted with respect to the one or more PCB 18 and wired with respect to ancillary components of the plug-in Wi-Fi access point device 10, such as, for example, to effect desired noise reduction and/or other electronic communication, transmission and/or processing functionalities.

With further reference to FIG. 1 and FIG. 2, the exemplary plug-in Wi-Fi access point device 10 in accordance with the present disclosure includes a connector 20 mounted on one side 22 of the device 10. For example, the connector 20 may be an RJ45 plug suitable for being directly plugged into an Ethernet jack of a wall or workstation having one or more RJ45 jacks. Exemplary RJ45 jacks include, but are not limited to, CAT5, CAT5E, CAT6, and CAT7. The plug-in feature of the plug-in Wi-Fi access point device 10 provides for ease of integration and eliminates the need for a patch cord to connect the Wi-Fi access point device 10 to a box or workstation.

With still further reference to FIG. 1 and FIG. 2, the exemplary plug-in Wi-Fi access point device 10 in accordance with the present disclosure may also optionally include a locking or self-locking mechanism 24 for securing the plug-in Wi-Fi access point device 10 into the face plate/workstation. The locking or self-locking mechanism 24 locks the connector 20 of the plug-in Wi-Fi access point device 10 into the Ethernet jack of a face plate or workstation. The locking or self-locking mechanism 24 may be unlocked via a specific tool or mechanism provided only to authorized users. The locking or self-locking mechanism 24 decreases the propensity for unauthorized tampering and theft of the plug-in Wi-Fi access point device 10 when in-use.

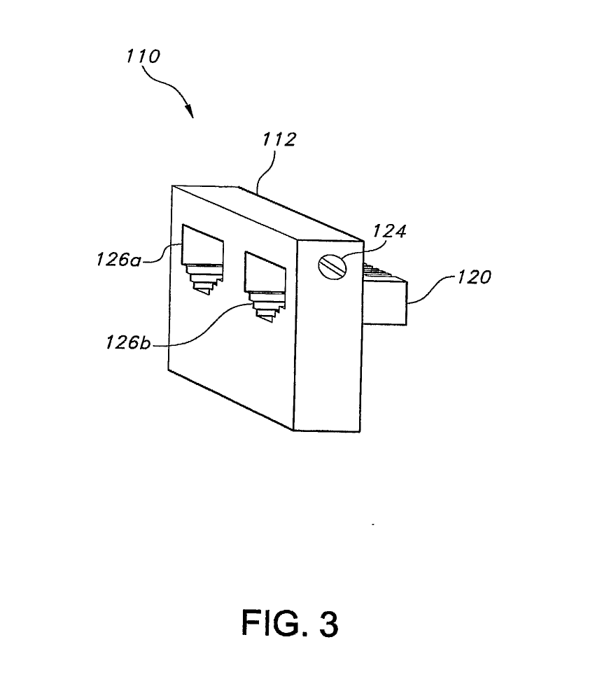

An alternative exemplary embodiment of the plug-in Wi-Fi access point device according to the present disclosure is schematically depicted in FIG. 3. The plug-in Wi-Fi access point device 110 is similar to the plug-in Wi-Fi access point device 10 described above, and therefore like reference numerals preceded by the numeral “1” are used to indicate like elements. The plug-in Wi-Fi access point device 110 includes a housing 112 suitable for accommodating Wi-Fi access point circuitry, one or more antennas (not shown) mounted with respect to one or more substrates (e.g., a predefined printed circuit board (PCB)) (not shown), an integrated hub/switch/router (not shown), a connector 120 mounted on one side of the device 110, and an optional locking or self-locking mechanism 124 for securing the plug-in Wi-Fi access point device 10 when in-use. The plug-in Wi-Fi access point device 110 is distinct from the exemplary embodiment of FIG. 1 and FIG. 2 in that the plug-in Wi-Fi access point device 110 also includes at least one integrated voice, video and/or data jack 126a, 126b for voice, video and/or data communications. For example, from 1 to 4 integrated data, video and/or voice jacks 126a, 126b, may be mounted within the housing 112 of the plug-in Wi-Fi access point device 110. The exemplary embodiment of FIG. 3 does not decrease the number of jacks available at the face plate/workstation when the plug-in Wi-Fi access point device 110 is in-use because of the additional voice, video and/or data jacks provided within the plug-in Wi-Fi access point device 110.

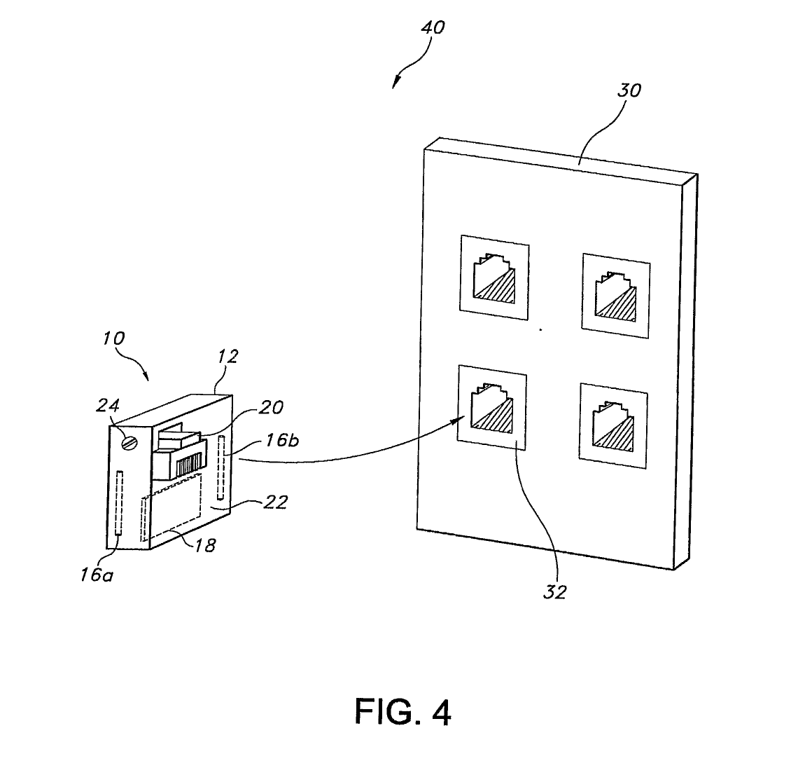

With reference to FIG. 4, there is shown by way of illustrative example a plug-in Wi-Fi access point system in accordance with the present disclosure generally represented by reference numeral 40. The Wi-Fi access point device 10 is the device previously described above and illustrated in FIGS. 1 and 2. The plug-in Wi-Fi access point device 10 includes a housing 12 suitable for accommodating Wi-Fi access point circuitry, one or more antennas 16a, 16b mounted with respect to one or more substrates 18 (e.g., a predefined printed circuit board (PCB)), a connector 20 mounted on one side 22 of the device 10, an optional integrated hub/switch/router (not shown), and optional locking or self-locking mechanism 24 for securing the plug-in Wi-Fi access point device 10 when in-use. The connector 20, for example a RJ45 plug, of the Wi-Fi access point device 10 mates or plugs-into the face plate/workstation 30. The face plate/workstation includes one or more RJ45 jacks 32, for example, CAT5, CAT5E, CAT6, and CAT7, suitable for receiving the connector 20 of the Wi-Fi access point device 10. The direct plug-in feature of the plug-in Wi-Fi access point device 10 into the face plate/workstation 30 eliminates the need for face plate removal and connection of a patch cord between the Wi-Fi access point device 10 and the workstation 30, which simplifies and expedites the installation process. Power is supplied to the plug-in Wi-Fi access point device 10 by a cable which supplies power over Ethernet, thereby drawing power for operation of Wi-Fi access point circuitry associated with the plug-in Wi-Fi access point device 10 from a network source. For example, the plug-in Wi-Fi access point device 10 may be powered via connectivity to the RJ45 jack 32 of the face plate/workstation 30 to obviate the need for a separate power source to power the plug-in Wi-Fi access point device 10.

With further reference to FIG. 4, the optional locking or self-locking mechanism 24 locks the connector 20 of the plug-in Wi-Fi access point device 10 into the RJ45 jack 32 of a face plate or workstation 30 to decrease the propensity for tapering and theft of the Wi-Fi access point device 10 while in-use. The locking or self-locking mechanism 24 may be unlocked via a specific tool or mechanism (not shown) provided to authorized users. Moreover, the plug-in Wi-Fi access point device 10 of FIG. 4 may be substituted with the plug-in Wi-Fi access point device 110 of FIG. 3 so as to provide one or more integrated voice, video and/or data jack 126a, 126b for voice, video and/or data communications. When one or more integrated voice, video and/or data jacks 126a, 126b for voice, video and/or data communications are integrated into the plug-in Wi-Fi access point device 110, the number of jacks available at the workstation when the plug-in Wi-Fi access point device 110 is in-use is not decreased.

With still further reference to FIG. 4, once connectivity is established between the plug-in Wi-Fi access point device 10 and the face plate/workstation 30 to establish the plug-in Wi-Fi access point system 40, a dedicated wireless network service is provided. A method of creating a plug-in Wi-Fi access point system 40 includes the steps of: 1) providing a plug-in Wi-Fi access point device 10, 110 that includes a housing 12, 112, Wi-Fi access point circuitry and one or more antenna 16, 116 in electronic communication with the Wi-Fi access point circuitry and a connector 20, 120, 2) providing a face plate/workstation 30 including at least one RJ45 jack 32 for network connectivity, 3) plugging the connector 20, 120 of the Wi-Fi access point device 10, 110 into the RJ45 jack 32 of the face plate/workstation 30, and 4) initializing the plug-in Wi-Fi access point device 10, 110 with the appropriate parameters for the wireless device to be connected to the wired network. The method of forming a plug-in Wi-Fi access point system 40 optionally includes further steps of: 1) providing a plug-in Wi-Fi access point device 10, 110 that further includes a locking or self-locking mechanism 24, 124 and at least one integrated voice, video and/or data jacks 126a, 126b for voice, video and/or data communications, 2) locking the plug-in Wi-Fi access point device 10, 110 into the face plate/workstation 30 by inserting the connector 20, 120 into the RJ45 jack 32, 132, and 3) establishing voice, video and/or data communications via connectivity to and at least one of the integrated voice, video and/or data communication jacks 126a, 126b.

In use, the Wi-Fi access point device and system provides for the removal of the patch cord that is typically required to connect the access point to the wired network by providing for a direct connection of the access point to the wired network via a connector integrated within the access point. The plug-in Wi-Fi access point device and system provides a discrete, visibly appealing access point with respect to the front of the wall or the front of existing face plate/workstation for plug-in, which is free from any exposed wired network ports or jacks so as to facilitate more efficient dimensioning and compact sizing of the overall assembly. Furthermore, the plug-in Wi-Fi access point device and system provides for improved ease of integration into an existing wired network, such for example, via an RJ45 jack mounted on an existing face plate or workstation without being obtrusive to adjacent ports of the existing face plate/workstation that it plugs into. Moreover, the plug-in Wi-Fi access point device and system is powered through an Ethernet connection so as to thereby avoid the need for a separate power source/supply.

Optionally in use, the Wi-Fi access point device and system provides for enhanced security and tamper resistance by allowing removal by only authorized personnel via the use of a specific tool or mechanism to unplug it from existing face plate/workstation. The plug-in Wi-Fi access point device and system optionally provides one or more integrated voice, video and/or data RJ45 jacks to maintain or augment the number of jacks available at the face plate/workstation. Still further, the plug-in Wi-Fi access point device and system has applicability for installations regardless of, and completely independent of, any applicable standards, whether now existing or developed in the future. Thus, the plug-in Wi-Fi access point devices and systems have applicability for any 802.11 network or other wireless networks, whether 802.11b, 802.11a, 802.11g, 802.16, dual-band, etc., but the disclosed devices/systems are expressly not limited to applications that conform to or satisfy such standards.

Although the plug-in Wi-Fi access point device and system of the present disclosure have been described with reference to exemplary embodiments, aspects and implementations thereof, the present disclosure is not limited to such. Rather, the present disclosure extends to and encompasses changes, modifications and/or variations thereof which will be apparent to persons skilled in the art based on the present disclosure, and such changes, modifications and/or variations are encompassed within the spirit and scope of the present disclosure.

1. A plug-in Wi-Fi access point device comprising:
a housing means for accommodating Wi-Fi access point circuitry;
a Wi-Fi access point circuitry mounted with respect to said housing means; and
a connector means mounted with respect to said housing means; wherein said connector means is dimensioned for direct plug-in into an Ethernet jack of a face plate/workstation for providing network connectivity.

2. The plug-in Wi-Fi access point device access point device of claim 1, further comprising at least one communication element mounted with respect to said housing means, wherein said at least one communication element is selected from the group consisting of: (i) one or more voice jacks, (ii) one or more video jacks, (iii) one or more data jacks, and (iv) a combination thereof.

3. The plug-in Wi-Fi access point device access point device of claim 1, further comprising a locking or self-locking means for securing said device within said face plate/workstation.

4. The plug-in Wi-Fi access point device access point device of claim 3, further comprising at least one communication element mounted with respect to said housing means, wherein said at least one communication element is selected from the group consisting of: (i) one or more voice jacks, (ii) one or more video jacks, (iii) one or more data jacks, and (iv) a combination thereof.

5. The plug-in Wi-Fi access point device access point device of claim 4, wherein said housing means is a rectangular shaped housing structure dimensioned to be unobtrusive to other jacks of said face plate/workstation when said connector means is plugged into a jack of said face plate/workstation.

6. The plug-in Wi-Fi access point device access point device of claim 5, wherein said connector means is mounted on one side of said rectangular shaped housing structure and said at least one communication element is mounted on an opposite side of said rectangular shaped housing structure.

7. The plug-in Wi-Fi access point device access point device of claim 6, wherein said housing means includes vent openings for venting heat generated within said rectangular shaped housing structure.

8. The plug-in Wi-Fi access point device of claim 1, wherein said Wi-Fi access point circuitry comprises one or more antenna, one or more printed circuit board, and an integrated hub/switch/router.

9. The plug-in Wi-Fi access point device of claim 8, wherein said one or more antenna includes a diversity antenna.

10. The plug-in Wi-Fi access point device of claim 1, wherein said connector means is an RJ45 plug.

11. The plug-in Wi-Fi access point device of claim 1, wherein said faceplate/workstation for providing network connectivity comprises one or more RJ45 jacks.

12. The plug-in Wi-Fi access point device of claim 1, wherein said Ethernet jack of said faceplate/workstation provides power to said device.

13. The plug-in Wi-Fi access point device of claim 1, further comprising electronic components mounted with respect to said substrate for effectuating noise reduction.

14. A plug-in Wi-Fi access point device comprising:
a housing for accommodating Wi-Fi access point circuitry,
Wi-Fi access point circuitry mounted within said housing, and
a connector mounted on a side of said housing for direct plug-in into an Ethernet jack of a face plate/workstation.

15. The plug-in Wi-Fi access point device of claim 14 further comprising a locking or self-locking mechanism for securing said connector within said Ethernet jack of said face plate/workstation.

16. The plug-in Wi-Fi access point device of claim 14 further comprising at least one communication element mounted with respect to said housing, wherein said at least one communication element is selected from the group consisting of: (i) one or more voice jacks, (ii) one or more video jacks, (iii) one or more data jacks, and (iv) a combination thereof.

17. The plug-in Wi-Fi access point device of claim 16 further comprising a locking or self-locking mechanism for securing said connector within said Ethernet jack of said face plate/workstation.

18. The plug-in Wi-Fi access point device of claim 14, wherein said Wi-Fi access point circuitry comprises one or more antenna, one or more printed circuit board, and an integrated hub/switch/router.

19. The plug-in Wi-Fi access point device of claim 14, wherein said connector on said housing is a RJ45 plug, and said faceplate/workstation for providing network connectivity comprises one or more RJ45 jacks.

20. A method of creating a Wi-Fi access point system comprising the following steps:
(a) providing a plug-in Wi-Fi access point device including a housing, Wi-Fi access point circuitry, and one or more antenna in electronic communication with the Wi-Fi access point circuitry, and a RJ45 plug mounted on said housing;
(b) providing a face plate/workstation including at least one RJ45 jack for network connectivity;
(c ) connecting said RJ45 plug mounted on said housing to said at least one RJ45 jack of said face plate/workstation; and
(d) initializing said plug-in Wi-Fi access point device with parameters for connecting a wireless device to said Wi-Fi access point system.

21. The method of creating a Wi-Fi access point system of claim 20,
wherein said step of providing a plug-in Wi-Fi access point device further includes the step of providing a locking or self-locking mechanism integrated into said housing, and
wherein said step of connecting said RJ45 plug mounted on said housing to said at least one RJ45 jack of said face plate/workstation locks said Wi-Fi access point device into said face plate/workstation.

22. The method of creating a Wi-Fi access point system of claim 20 further comprising the steps of:
(a) providing at least one communication element mounted with respect to said housing; and
(b) connecting one or more voice, video or data communications devices to said at least one communication element for voice, video and data communications;
wherein said at least one communication element is selected from the group consisting of: (i) one or more voice jacks, (ii) one or more video jacks, (iii) one or more data jacks, and (iv) a combination thereof.
